(19) United States
(12) Patent Application Publication
(10) Pub. No.: US 2012/0129188 A1
(43) Pub. Date: May 24, 2012

(54) CIRCULATING CYTOCHROME C AS BIOMARKER OF REPERFUSION INJURY AND RESPONSIVENESS TO MITOCHONDRIAL TARGETED INTERVENTIONS

(75) Inventor: Raul J. Gazmuri, Chicago, IL (US)

(73) Assignee: Rosalind Franklin University of Medicine and Science, North Chicago, IL (US)

(21) Appl. No.: 13/289,686

(22) Filed: Nov. 4, 2011

Related U.S. Application Data
(63) Continuation-in-part of application No. 12/613,919, filed on Nov. 6, 2009, now Pat. No. 8,067,366.
Application No. 12/613,919 is a continuation-in-part of application No. 11/489,846, filed on Jul. 20, 2006, now Pat. No. 8,133,860.
(60) Provisional application No. 61/410,255, filed on Nov. 4, 2010.

(51) Int. Cl. G01N 33/566; G01N 30/00

(52) U.S. Cl. 435/7.9; 73/61.52

(57) ABSTRACT

The present invention relates generally to the use of circulating cytochrome c as a biomarker of reperfusion injury that results from whole body ischemia. Circulating levels of cytochrome c can be used as predictor of survival rates and to assess the effects of interventions aimed at ameliorating mitochondrial injury during reperfusion. Whole body ischemia may be the result of cardiac arrest or from other hemodynamic crises, such as hemorrhagic shock.

### CROSS REFERENCE TO RELATED APPLICATIONS

The present application is a continuation-in-part of U.S. patent application Ser. No. 12/613,919 filed on Nov. 6, 2009 entitled “Facilitation of Resuscitation from Cardiac Arrest by Erythropoietin” which is a continuation-in-part of U.S. patent application Ser. No. 11/489,846 filed on Jul. 20, 2006 with the same title and also claims priority to U.S. Provisional PatentApplication No. 61/410,255 filed on Nov. 4, 2010 and entitled “Circulating Cytochrome C as Biomarker of Reperfusion Injury and Responsiveness to Mitochondrial Targeted Interventions” all of these applications are incorporated in their entirety herein by reference and made a part hereof.

### BACKGROUND OF THE INVENTION

1. Field of the Invention

The present invention relates generally to the use of circulating cytochrome c as a biomarker of reperfusion injury and of responsiveness to mitochondrial targeted interventions given during resuscitation after whole body ischemia. Whole body ischemia may be the result of cardiac arrest or other low-flow states, including conditions that cause reduction in circulating blood volume such as hemorrhagic and hypovolemic shock.

2. Background of the Invention

Critical reduction in whole body oxygen and energy substrates delivery leading to organ ischemia occurs after cardiac arrest and other low-flow states precipitated by severe reduction in circulating blood volume. Reperfusion during resuscitation from cardiac arrest and the aforementioned low-flow states attempts to restore cell viability and cell function by delivering oxygen and energy substrates. However, reperfusion also triggers a complex response leading to cell injury despite adequate return of oxygen and energy substrates. This type of injury is known as reperfusion injury and manifests at the organ level by variable degrees of organ dysfunction that may compromise survival despite reversal of the precipitating event. At the center of reperfusion injury are mitochondria, playing a critical role as effectors and targets of injury. Calcium overload and reactive oxygen species are main events that cause mitochondrial injury during ischemia and reperfusion.

Novel approaches are needed to enable real-time recognition, severity assessment, and treatment of reperfusion injury. Recent studies demonstrate that attenuation of mitochondrial injury during cardiac resuscitation is achieved by administering high-dose erythropoietin (EPO) during resuscitation from cardiac arrest. The effect of EPO manifests, at least in part, by preservation of left ventricular myocardial distensibility during cardiac resuscitation and by attenuation of post-resuscitation myocardial dysfunction. These findings are discussed in detail in U.S. patent application Ser. Nos. 11/489,846 and 12/613,919. The present invention confirms that mitochondrial injury causes cytochrome c to release into the cytosol and bloodstream while activating apoptotic pathways during cardiac resuscitation. The present invention discloses that measuring circulating levels of cytochrome c after resuscitation from cardiac arrest predicts subsequent survival. The present invention further discloses that administration EPO for attenuating mitochondrial injury results in higher activity of cytochrome c oxidase in left ventricular tissue, increased integrity of the outer mitochondrial membrane, and better post-resuscitation left ventricular and hemodynamic function and that these effects are associated with lower plasma levels of cytochrome c. These observations support the concept that mitochondrial injury caused by cardiac arrest and reperfusion leads to myocardial dysfunction and to hemodynamic dysfunction which are in part responsible for reduced survival. Circulating levels of cytochrome c serve to identify these abnormities, recognize the effects of interventions, such as EPO, that can attenuate mitochondrial injury, and predict survival. Consequently, the present invention is directed at measuring plasma levels of cytochrome c in a subject as a novel biomarker of mitochondrial injury useful to assess responsiveness to mitochondrial targeted interventions, such as but not limited to EPO, delivered for the purpose of attenuating reperfusion injury during resuscitation from cardiac arrest and other low-flow states.

These and other aspects and attributes of the present invention will be discussed with reference to the following drawings and accompanying specification.

### SUMMARY OF THE INVENTION

The present invention relates generally to the use of circulating cytochrome c as a biomarker of mitochondrial injury for assessing responsiveness to mitochondrial targeted interventions delivered to attenuating reperfusion injury during resuscitation from whole body ischemia. As such, circulating levels of cytochrome c correlates with left ventricular and hemodynamic function and serves to predict survival following resuscitation from cardiac arrest and other low-flow states while identifying mitochondrial interventions that attenuate reperfusion injury and could improve outcome.

One embodiment of the present invention discloses a method for determining severity of reperfusion injury after resuscitation in a mammalian subject having suffered from whole body ischemia wherein the method comprises measuring the plasma level of cytochrome c in the mammalian subject post-resuscitation and comparing those levels to a normal level of cytochrome c of the mammalian subject, wherein an increase in the plasma cytochrome c level post-resuscitation compared to the normal level is indicative of reperfusion injury in the subject. The whole body ischemia may be due to cardiac arrest or other low-flow states, including conditions causing severe reduction in circulation blood volume such as hemorrhagic and hypovolemic shocks.

In another embodiment, the present invention discloses a method for predicting the survival rates of a mammalian subject, wherein the plasma level of cytochrome c in the mammalian subject is measured over a period of time after the resuscitation, wherein a continuous increase in the plasma cytochrome c level after the resuscitation compared to a normal level is indicative of low survivability of the subject, and a return of the plasma cytochrome c level to the normal level indicates high survivability of the subject.

In another embodiment, the present invention further discloses the effects of EPO in a mammalian subject when administered during resuscitation from cardiac arrest. This embodiment demonstrates that administration of EPO at the time of resuscitation results in attenuation of left ventricular mitochondrial injury associated with better left ventricular function and reduced circulating levels of cytochrome c. The mammalian subject is preferably a human subject.

### BRIEF DESCRIPTION OF THE DRAWINGS

FIG. 1 shows the results of plasma cytochrome c measured by HPLC. A: Standard curve using rat heart cytochrome c dissolved in plasma obtained from pentobarbital-anesthetized rats not subject to surgical manipulation. Absorbance was measured at 393 nm (ambient temperature) and the peak area determined by integrating the beginning to the end of the chromatographic peak center at ≈9.5 minutes retention time (arrow in panels B and C). Solid line represents linear fit of the data (r²=0.9996). B: Chromatographs of plasma obtained before and after right atrial injection of cytochrome c (5 mg/kg) during spontaneous circulation in a 500 g rat demonstrating capability for detecting plasma cytochrome c. C: Representative chromatographs of plasma obtained in a 506 g rat resuscitated after 8 minutes of untreated VF. The rat died at 8.5 hours post-resuscitation (PR) preceded by progressive rise in plasma cytochrome c;

FIG. 2 shows the results of hemodynamic and left ventricular measurements at baseline and post-resuscitation in rats randomized to untreated ventricular fibrillation (VF) lasting 4 minutes (closed gray circles, n=4), 8 minutes (closed black circles, n=4), or to sham intervention (open circles, n=4). Numbers in brackets indicate rats still alive. BL=Baseline; LVSWI=Left ventricle stroke work index; CI=Cardiac index; MAP=Mean aortic pressure. The shaded horizontal bar across each graph represents the 95% confidence interval of the aggregate baseline values. Mean±SEM. *p<0.05; †p<0.01, and [symbol] p<0.001 vs sham by one-way ANOVA and Holm-Sidak's test for multiple comparisons;

FIG. 3 shows plasma cytochrome c (measured by HPLC), lactate, and bicarbonate (HCO₃⁻) at baseline, chest compression (for cytochrome c), and post-resuscitation in rats randomized to untreated VF lasting 4 minutes (closed gray circles, n=4), 8 minutes (closed black circles, n=4), or to sham intervention (open circles, n=4). Numbers in brackets indicate rats still alive. BL=Baseline; CC=Chest compression. The shaded horizontal bar across each graph represents the 95% confidence interval of the aggregate baseline values. Mean±SEM. *p<0.05; †p<0.01 and [symbol] p<0.001 vs sham by one-way ANOVA and Holm-Sidak's test for multiple comparisons;

FIG. 4 shows the result of Western immunoblots of plasma cytochrome c using rabbit polyclonal anti-cytochrome c primary antibody (1:2000 dilution) and goat polyclonal anti-rabbit IgG HRP conjugated secondary antibody (1:1000 dilution) in three representative experiments. Eight μl of plasma were loaded in each lane. Plasma cytochrome c levels remained unchanged in sham controls but progressively rose in rats subjected to VF. Negative control (nc) was obtained from a cytochrome c positive rat plasma (240 minutes post-resuscitation) in which the primary antibody was omitted. Positive control (pc) represents the antibody reactivity to rat heart cytochrome c from Sigma;

FIG. 5 shows the result of Western immunoblots of prohibitin (30 kDa) and cytochrome c (14 kDa) in left ventricular cytosolic (c) and mitochondrial (m) fractions (20 μg each) from 2 rats each randomized to untreated VF lasting 4 minutes (VF 4-mins), 8 minutes (VF 8-mins), or to sham intervention. Prohibitin served as loading control for the mitochondrial fraction and to exclude mitochondrial contamination of the cytosolic fraction. β-actin (42 kDa) served as loading control for the cytosolic fractions. Negative controls (nc) obtained by omitting the primary antibody. B: Western immunoblots of caspase-3, full-length (35 kDa) and cleaved fragments (19 and 17 kDa) in left ventricular cytosolic fraction (100 μg) from 2 rats each randomized to untreated VF lasting 4 minutes, 8 minutes, or to sham intervention. β-actin (42 kDa) served as loading control. Negative controls (nc) obtained by omitting the primary antibody. Longer exposure times were required to detect the cleaved fragments.

FIG. 6 is the result of densitometry of immunoblots demonstrating numerical increases in mitochondrial cytochrome c relative to prohibitin and cytosolic cytochrome c relative to β-actin and statistically significant increases in 17 kDa cleaved caspase-3 fragments in the cytosolic fraction relative to pro-caspase-3 and β-actin at 240 minutes post-resuscitation. Rats were randomized to untreated VF lasting 4 minutes (closed gray bars, n=4), 8 minutes (closed black bars, n=4), or to sham intervention (open bars, n=4). Mean±SEM. *p<0.05 vs sham by one-way ANOVA and Dunn's test for multiple comparisons;

FIG. 7 is the result of serial measurements of plasma cytochrome c by reverse-phase HPLC in rats successfully resuscitated after 8 minutes of untreated VF. Measurements were made until the cytochrome c level had returned to baseline or the rat had died. Open circles and open bars represent survivors (n=3) and closed circles and closed bars represent non-survivors (n=9);

FIG. 8 demonstrates the effects of erythropoietin (open circles) compared with vehicle control (closed circles) on post-resuscitation hemodynamic and left ventricular function. Each group is represented by 5 rats. HR=heart rate, MAP=mean aortic pressure, SVI=stroke volume index, and CWI=cardiac work index. The values are shown as mean±SEM. Differences between groups were analyzed by two-way repeated measures ANOVA, examining the effects of time (repeated factor), treatment, and their interaction. *denotes p<0.005 between groups.

FIG. 9 demonstrates cytochrome c oxidase (left panel) and percentage of mitchondria with intact outer mitochrondial membrane (right panel) in left ventricular tissue of heart removed at 120 minutes post-resuscitation in 10 rats treated with erythropoietin (EPO) or vehicle control (saline).

FIG. 10 demonstrates cytochrome c (cyto c) and cardiac troponin I (cTn I) levels (ng/ml) in plasma at baseline (BL) and post-resuscitation (PR) at 60 and 120 minutes in rats treated with erythropoietin (open bars) and vehicle control (closed bars). Values are mean±SEM. N=5 for all but 4 for control cTn I at PR 120 minutes since technical errors precluded the measurement in one rat. *p=0.016 and **p=0.014 vs. control by Student's t-test; and

FIG. 11 demonstrates scatterplots depicting the correlations and linear regressions between plasma cytochrome c and mean aortic pressure (MAP), cardiac work index (CWI), cardiac index (CI), and stroke volume index (SVI) at post-resuscitation 120 minutes in 10 rats treated with erythropoietin (∘) or vehicle control ().

### DETAILED DESCRIPTION OF THE INVENTION

While this invention is susceptible of embodiments in many different forms, there are specific embodiments which will be described herein in detail with the understanding that the present disclosure is to be considered as an exemplification of the principles of the invention and is not intended to limit the invention to the specific embodiments illustrated.

The present invention relates generally to the use of circulating cytochrome c in the blood, with measurements in this embodiment made in plasma, of a mammalian subject as a biomarker of reperfusion injury resulting from whole body ischemia and predictor of responsiveness to mitochondrial targeted interventions, such as but not limited to EPO, and of survival following reperfusion injury that occurs coincident with the resuscitation procedures. The present invention discloses that cytochrome c is a biomarker of reperfusion injury based on evidence establishing mitochondria at the center of ischemia and reperfusion injury, and the release of cytochrome c to the cytosol and bloodstream as the hallmark of such injury.

Cytochrome c is a 14-kDa hemoprotein that normally resides in the inter-membrane mitochondrial space bound to cardiolipin, and is essential for oxidative phosphorylation by enabling electron transfer from cytochrome c reductase to cytochrome c oxidase. Release of cytochrome c to the cytosol may therefore disrupt oxidative phosphorylation and compromise the generation of ATP by mitochondria. In addition, cytochrome c in the cytosol activates the intrinsic apoptotic pathway by cleavage of functional and structural proteins of the apoptotic pathway initiating programmed cell death. Increased plasma levels of cytochrome c, inversely related to outcomes, have been reported in patients with the systemic inflammatory response syndrome, influenza-associated encephalopathy, and fulminant hepatitis.

The present invention discloses the effects of ischemia and reperfusion injury during resuscitation from cardiac arrest on cytosolic and circulating levels of cytochrome c in a rat model of ventricular fibrillation (VF) and closed-chest resuscitation. The studies demonstrate that cytochrome c is released to the cytosol after resuscitation from VF in association with caspase-3 activation and left ventricular dysfunction and that cytochrome c is then released to the bloodstream where it can be detected through several methods. The present invention further discloses that interventions, such as EPO, that attenuate mitochondrial injury during reperfusion lead to improved left ventricular and hemodynamic function and lower circulating levels of cytochrome c. The present invention uses HPLC and Western immunoblotting for detecting circulating levels of cytochrome c. Examples of methods using HPLC and immunoblotting to measure circulating cytochrome c levels are disclosed in the examples below along with examples of uses to assess left ventricular and hemodynamic function, effects on survival, and effects of interventions targeting mitochondria such as EPO.

### EXAMPLES

### Materials Used in Examples

Acetonitrile, ethylene diamine tetraacetic acid (EDTA), (N-[2-hydroxyethyl]piperazine-N′-[2-ethanesulfonic acid]) (HEPES), mannitol, propidium iodide (PI), rat heart cytochrome c, sucrose, trifluoro-acetic acid, and mouse-monoclonal anti-β-actin antibody were purchased from Sigma; 1-mm thick 12% and 14% Novex® tris-glycine polyacrylamide gels from Invitrogen Corporation; polyvinylidene difluoride (PVDF) membrane from Roche Applied Science; rabbit polyclonal anti-cytochrome c, and rabbit polyclonal anti-caspase-3 antibodies from Cell Signaling Technology; mouse monoclonal anti-prohibitin antibody from Calbiochem; annexin V conjugated with fluorescein isothiocyanate (FITC) and mouse-monoclonal anti-cytochrome c antibody (clone 7H8.2C12) were from BD Biosciences PharMingen; Immunolyse (red blood cell lysis agent) from Coulter; and goat polyclonal anti-rabbit IgG along with goat polyclonal anti-mouse IgG horseradish peroxidase (HRP) conjugated antibodies, bicinchoninic acid (BCA) protein concentration assay kit, and West femto maximum sensitivity chemiluminescent detection kit from Pierce Biotechnology Inc.

### Statistical Analysis Used in Examples

Differences among groups for continuous variables were analyzed using one-way ANOVA and Holm-Sidak's for multiple comparisons. Alternative non-parametric tests were used if the data failed tests for normality or equal variance. The strength of association between variables was analyzed using Pearson's product moment correlation test. The data were presented as mean±SD unless otherwise stated. A two-tail value of p<0.05 was considered significant.

### Example 1

### Animal Preparation for VF and Resuscitation Protocol

Adult male Sprague-Dawley rats (480 to 550 g) were anesthetized using sodium pentobarbital (45 mg/kg intraperitoneal for induction and 10 mg/kg intravenous every 30 minutes for maintenance). A 5-F cannula was orally advanced into the trachea and used for positive pressure ventilation during cardiac resuscitation and the post-resuscitation interval. Proper placement was verified using an infrared CO₂ analyzer (CO₂SMO model 7100, Novametrix Medical Systems, Inc.). A conventional lead II ECG was recorded through subcutaneous needles. PE50 catheters were advanced through the right femoral vein into the right atrium, from the right carotid into the left ventricle, and from the left femoral artery into the abdominal aorta for pressure measurement and blood sampling. A thermocouple microprobe (IT-18, Physitemp) was advanced through the right femoral artery into the thoracic aorta for thermodilution cardiac output measurement. A PE50 catheter was advanced through the left external jugular vein into the right atrium and used exclusively for injection of thermal tracer. A 3-F catheter (C-PUM-301J, Cook, Inc.) was advanced through the right external jugular vein into the right atrium, and through its lumen a pre-curved guide wire fed into the right ventricle and used for electrical induction of VF. Core temperature was maintained between 36.5° C. and 37.5° C. using an infrared heating lamp.

VF was induced by delivering a 60-Hz alternating current to the right ventricular endocardium (0.1 to 0.6 mA) for an uninterrupted interval of three minutes after which the current was turned off and VF allowed to continue until completion of a predetermined interval (see below). Chest compression was then begun using an electronically controlled and pneumatically driven (50 PSI) chest compressor (CJ-80623, CJ Enterprises) set to deliver 200 compressions/min with a 50% duty cycle. The depth of compression was adjusted to attain an aortic diastolic pressure between 26 and 28 mm Hg ensuring a coronary perfusion pressure above the resuscitability threshold of 20 mm Hg in rats. Positive pressure ventilation was provided using an electronically controlled solenoid valve (R-481, Clippard Instrument Laboratory Inc.) set to deliver 0.39 mL/100 g body weight of 100% oxygen every two compressions. Defibrillation was attempted after 8 minutes of chest compression by delivering a maximum of two 2-J monophasic transthoracic shocks (Lifepak 9P, Physio-Control). If VF persisted or an organized rhythm with a mean aortic pressure of ≦25 mm Hg ensued, chest compression was resumed for 30 seconds. The defibrillation-compression cycle was repeated up to three additional times, increasing the energy of individual shocks if VF persisted to 4-J and then to 8-J for the last two cycles. Successful resuscitation was defined as the return of an organized cardiac rhythm with a mean aortic pressure ≧60 mmHg for ≧5 minutes. Resuscitated rats were ventilated initially with 100% O₂ for 15 minutes and then continued with 50% O₂ for the remaining post-resuscitation interval.

### Example 2

### Hemodynamic Variables

Continuous physiological measurements were transduced, conditioned (BIOPAC Systems), and digitized at 250 scans per second using a 16-bit data acquisition board (AT-MIO-16XE-50; National Instruments). Systemic and left ventricular pressures were obtained through fluid-filled systems attached to disposable pressure-transducers (Maxxim Medical) zeroed to midchest level. Cardiac output was measured by thermodilution after right atrial bolus injection of 200 μl of 0.9% NaCl at room temperature and curve-analysis using custom-developed LabVIEW-based software. Cardiac index (CI) was calculated by normalizing cardiac output in ml/min to body weight in kilograms. The stroke volume index (SVI) was calculated by dividing cardiac index by heart rate. Left ventricular stroke work index (LVSWI) was calculated by multiplying the SVI by the difference between left ventricular systolic and diastolic pressures.

### Example 3

### Measurement of Plasma Cytochrome c

Arterial blood samples (200 μl) were collected into heparinized syringes and centrifuged at 5,000 rpm (2,320 g) for 10 minutes at 4° C. The supernatant (plasma) was frozen at −80° C. for differed analysis using reverse-phase high performance liquid chromatography (HPLC) and western immunoblotting in series-1 but used immediately for analysis by HPLC in series-2.

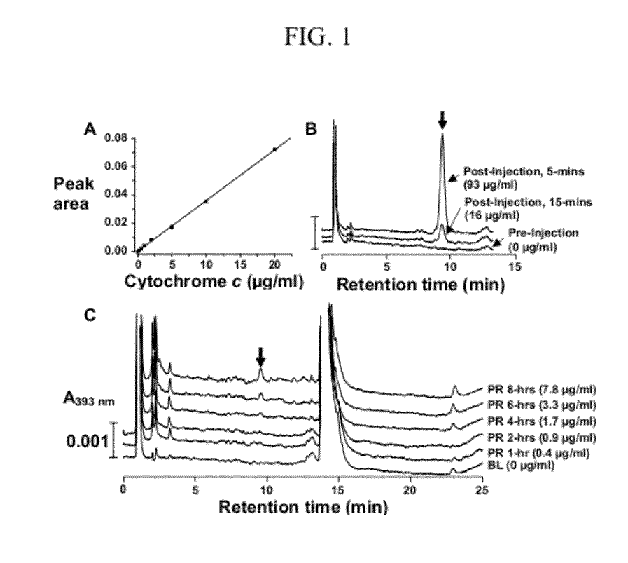

HPLC: A reverse-phase HPLC technique previously used to measure cytochrome c in mitochondrial suspension and cytosol was adapted for measuring cytochrome c in plasma. Samples were first treated with 50% acetonitrile solution containing 0.1% trifluoro-acetic acid (ACN-TFA) (1:1, V/V) and then centrifuged at 5,000 rpm (2,320 g) for 10 minutes to precipitate plasma proteins of high molecular weight. Cytochrome c was measured in the supernatant using a Beckman HPLC System equipped with a Jupiter C4 reverse-phase analytical column (150×4.6 mm, 5 μm, Phenomenex) preceded by a guard column (SecurityGuard, 4.0×3.0 mm, widepore C4 [Butyl], Phenomenex), an ultraviolet-visible light spectroscopic detector, and a Beckman System Gold software. The mobile phase was programmed to provide a constant-flow (1.0 ml/min) linear gradient starting at 20% ACN-TFA and increasing to 60% ACN-TFA over a 15-minute interval followed by a 5-minute 60% ACN-TFA wash and a 5-minute 20% ACN-TFA equilibration between samples. Each sample (20 μl) was loaded in an injection loop and automatically delivered into the mobile phase at the start of the linear gradient. Absorbance was measured at 393 nm. For quantification of plasma cytochrome c levels, standard curves (0.2 to 20 μg/ml) were prepared using rat heart cytochrome c dissolved in plasma obtained from pentobarbital-anesthetized rats not subject to surgical manipulation (FIG. 1, panel A). Capability for detection of in vivo increases in cytochrome c was confirmed by serial plasma measurements after injection of 5 mg/kg of rat heart cytochrome c during spontaneous circulation (FIG. 1, panel B).

### Example 4

### Western Immunoblotting

Plasma samples were thawed on ice and 8 μl resolved in 1-mm thick 12% Novex® tris-glycine polyacrylamide gel. Following electrophoresis, fractionated proteins were transferred to PVDF membrane using a wet electroblotting apparatus (Hoefer TE22, Amersham Pharmacia Biosciences). After transfer, the blots were blocked and incubated at 4° C. overnight with rabbit polyclonal anti-cytochrome c antibody (1:2000 dilution) followed by goat polyclonal anti-rabbit IgG HRP conjugated antibody (1:1000 dilution) for 1 hour at room temperature. Chemiluminescence was documented in X-ray film. Protein molecular weight markers were run simultaneously in each gel. The specificity of the cytochrome c band was based on molecular weight along with positive and negative controls.

Western Immunoblotting of Left Ventricular Tissue

Hearts from series-1 experiments were harvested at 240 minutes post-resuscitation or earlier if hemodynamic deterioration occurred with a decrease in the mean aortic pressure below 40 mmHg (two instances). The right ventricle and atria were removed and the left ventricle frozen in liquid N₂ and stored at −80° C. until analysis. For assessing cytochrome c release, subcellular fractions were separated by the technique of Ott et al. with minor modifications and subjected to western immunoblotting. Briefly, ≈100 mg of left ventricular tissue was homogenized in 2 ml MSH buffer (210 mM mannitol, 70 mM sucrose, 5 mM HEPES, pH 7.4, and 1 mM EDTA, pH 8) using a Dounce homogenizer and centrifuged at 1000 g for 10 minutes to pellet nuclei and debris. The resulting supernatant was then centrifuged at 10,000 g for 30 minutes. The pellet containing mitochondria was washed and resuspended in MSH buffer. The supernatant was further centrifuged at 33,000 g for 3.3 hours and the resulting supernatant collected representing the cytosolic fraction. Both fractions were stored at −80° C. until analysis. The protein concentration in each fraction was determined by the BCA kit. Mitochondrial and cytosolic fractions (20 μg each) were resolved in 1-mm thick 12% Novex® tris-glycine polyacrylamide gel and immunoblotted as described above for plasma cytochrome c. Cytochrome c was probed using a mouse monoclonal anti-cytochrome c antibody (1:4000 dilution). Prohibitin, a 30 kDa inner mitochondrial membrane protein, and β-actin were selected as loading controls for mitochondrial and cytosolic fractions respectively. Prohibitin was probed using a mouse monoclonal anti-prohibitin antibody (1:1000 dilution). β-actin was probed using a mouse monoclonal anti-β-actin antibody (1:2500 dilution). The blots were incubated with the primary antibodies for 3 hours at room temperature followed by incubation with goat polyclonal anti-mouse IgG HRP conjugate (1:1000 dilution) for 1 hour at room temperature and documented by chemiluminescence.

### Example 5

### Assessing Caspase-3 Activation

For assessing caspase-3 activation, 100 mg of left ventricular tissue was homogenized in 0.4 ml of MSH buffer with a mixer mill homogenizer (Mixer mill, MM200, Retsch GmbH & Co). The cytosolic fraction (100 μg) was separated by subcellular fractionation as described above and subjected to 14% gel electrophoresis and western immunoblotting using rabbit polyclonal anti-caspase 3 antibody (1:1000 dilution). The antibody recognizes 35 kDa procaspase-3 and its 19 and 17 kDa cleaved fragments. For loading controls, β-actin was used as described above.

### Example 6

### Experiments for Studying Cytochrome c Levels Post-Resuscitation

Two series of experiments were conducted. In series-1, changes in plasma cytochrome c were measured for up to 240 minutes post-resuscitation in fully instrumented rats as described above. In series-2, changes in plasma cytochrome c were measured for up to 96 hours post-resuscitation in rats that were minimally instrumented to facilitate survival as described below.

Series-1: Three groups of 4 rats each were investigated. Two groups were subjected to either 4 or 8 minutes of untreated VF followed by 8 minutes of closed-chest resuscitation before attempting defibrillation and observed for a maximum of 240 minutes post-resuscitation. One group served as sham and received identical treatment except for VF and closed-chest resuscitation. Rats were randomized to the experimental assignment immediately after completion of surgical preparation.

Series-2: The animal preparation differed from series-1 in that hemodynamic measurements were limited to the right atrial and aortic pressures. Thus, PE50 tubing was advanced from the left jugular vein into the right atrium and from left carotid artery into the descending thoracic aorta avoiding additional surgical manipulation except for the 3-F catheter (C-PUM-301J, Cook, Inc.) and guide wire required for induction of VF. Core temperature was measured through a rectal thermistor. During closed-chest resuscitation, rats were ventilated using a volume controlled ventilator (Model 683, Harvard Apparatus) set to deliver 6 ml/kg at 25 breaths/min unsynchronized to chest compression. For defibrillation, a biphasic waveform defibrillator (Heartstream XL, Philips Medical Systems) was used delivering 3-, 5-, and 7-J as needed according to series-1 protocol. These changes reflected implementation of new developments in cardiac resuscitation. After 240 minutes post-resuscitation rats were allowed to recover from anesthesia. The left carotid PE50 tubing was then replaced by soft PE50 tubing primed with heparinized glycerol. The proximal end was sealed with a metal plug that could be removed for blood sampling and tunneled underneath the skin to the interscapular region where a skin incision held closed by a removable metal clip enabled intermittent access. For blood sampling, rats were anesthetized using isoflurane (0.5 to 1.0%) and 100% oxygen collecting 200 μl of arterial blood into a heparinized syringe. A total of 12 rats were successfully resuscitated after 8 minutes of untreated VF and 8 minutes of closed-chest resuscitation. Cytochrome c was serially measured until the rat had died or cytochrome c had returned to baseline levels.

### Results in these Examples

Series-1

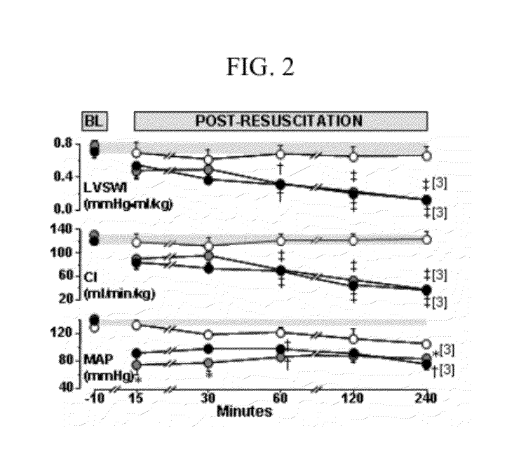

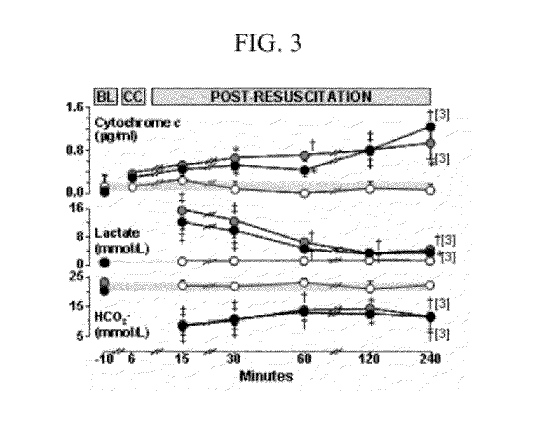

Baseline hemodynamic measurements were comparable among groups. Post-resuscitation, rats subject to VF developed characteristic post-resuscitation myocardial dysfunction evidenced by a persistently low mean aortic pressure and progressive reductions in LVSWI and CI without measurable differences related to the duration of untreated VF (FIG. 2). Myocardial dysfunction accounted for early termination of one rat subjected to 4 minutes of untreated VF (at 150 minutes) and one rat subjected to 8 minutes of untreated VF (at 155 minutes). As previously reported, cardiac arrest and resuscitation was associated with anaerobic metabolism evidenced by prominent increases in arterial lactate and reductions in arterial bicarbonate. Arterial lactate was maximal upon return of spontaneous circulation and gradually decreased towards baseline during the post-resuscitation interval (FIG. 3).

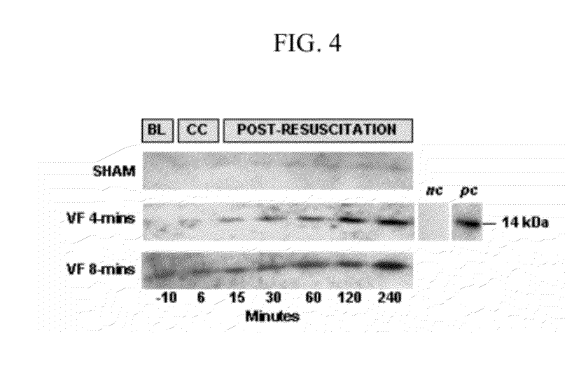

Plasma cytochrome c rose progressively during the post-resuscitation interval in rats subject to VF separating statistically from sham rats at 30 minutes post-resuscitation and onwards (FIG. 3). Western immunoblotting confirmed elevated levels of cytochrome c in plasma during the post-resuscitation interval (FIG. 4). The plasma levels of cytochrome c were inversely correlated with LVSWI (r=−0.40, p=0.02, for 38 measurements obtained at five post-resuscitation events in eight resuscitated rats).

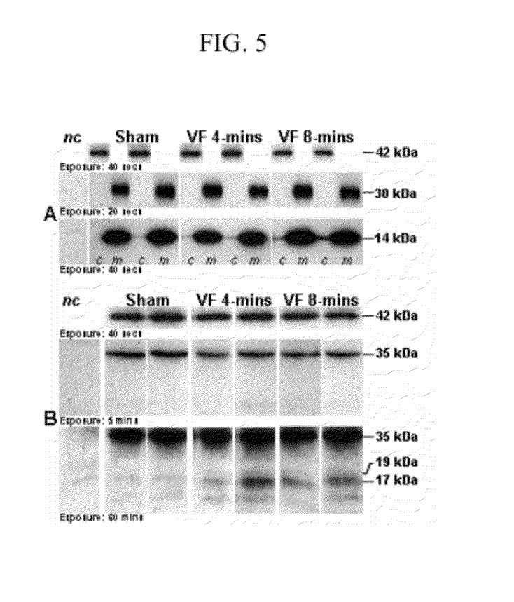

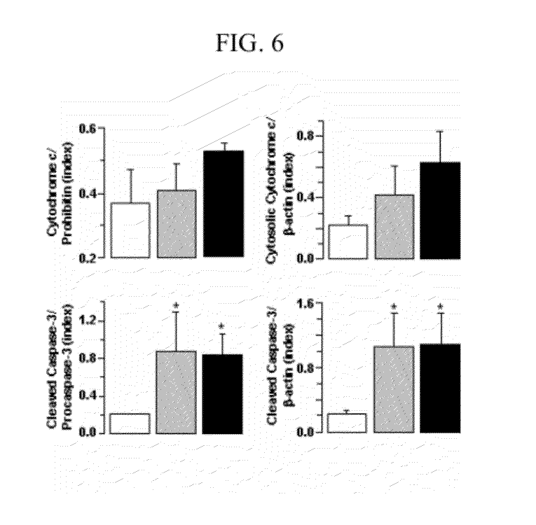

In left ventricular tissue, higher values for cytochrome c were observed in cytosolic and mitochondrial fractions proportional to the injury but the differences were not statistically significant (FIG. 5, panel A and FIG. 6). The level of 17 kDa caspase-3 fragments measured in the cytosolic fraction, however, was significantly increased in rats resuscitated from VF (FIG. 5, panel B and FIG. 6).

Series-2

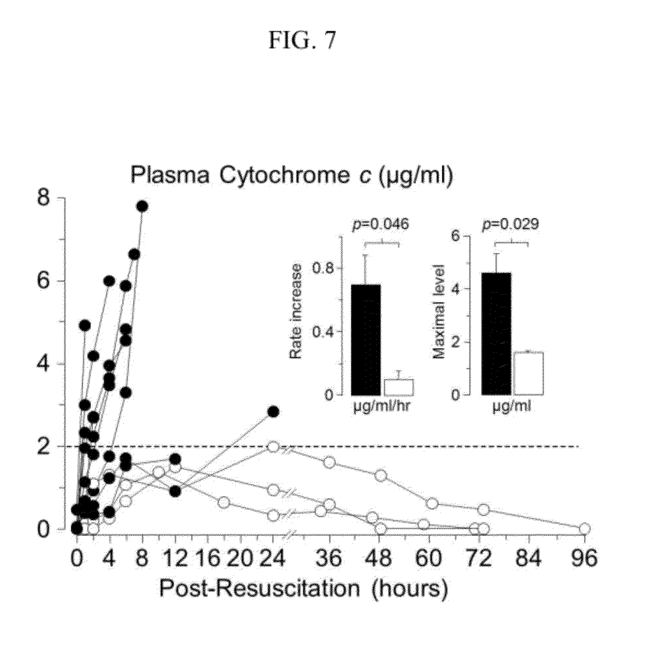

Of 12 successfully resuscitated rats, 3 survived for more than 24 hours and were euthanized according to protocol after 70 to 96 hours. In this group, cytochrome c rose gradually to levels not exceeding 2 μg/ml and then returned to baseline levels (FIG. 7). The remaining 9 rats died between 1.3 and 32.5 hours post-resuscitation having a progressive rise in plasma cytochrome c (FIG. 1, panel C and FIG. 7) attaining higher maximal levels (4.6±2.0 vs 1.6±0.3 μg/ml; p=0.029) and at faster rates (0.7±0.5 vs 0.1±0.05 μg/ml/hr; p=0.046) than in survivor rats. Comparison of survivors and non-survivors demonstrated similar coronary perfusion pressures during chest compression but numerically higher energy requirements for successful defibrillation in non-survivors (Table 1). Post-resuscitation, non-survivor rats had a numerically lower mean aortic pressure at 60 minutes (78±26 and 98±2 mmHg) and at 120 minutes (85±18 and 100±4 mmHg).

TABLE 1
Resuscitation effort

| | | Energy of initial | | Cumulative | |
|---|---|---|---|---|---|
| | Averaged CPP | successful shock | Total # of DF | DF energy | Duration of |
| | (mmHg) | (Joules) | shocks (n) | (Joules) | VF (Seconds) |
| Non-Survivors (n = 9) | 21 ± 3 | 4 ± 2 | 1.9 ± 1.5 | 7 ± 7 | 983 ± 29 |
| Survivors (n = 3) | 20 ± 3 | 3 ± 0 | 1.0 ± 0.0 | 3 ± 0 | 906 ± 103 |

CPP = Coronary perfusion pressure averaged from minute 2 to minute 8 of chest compression;
DF = Defibrillation;
VF = Ventricular fibrillation.
No statistical differences were observed between groups.

Thus, the evidence indicates that despite initial hemodynamic stabilization after identical levels of injury (cardiac arrest and resuscitation), a dichotomous outcome follows in which some subjects die and others survive. Consequently, the present invention demonstrates that this dichotomy can be identified with high sensitivity and specificity by measuring circulating plasma levels of cytochrome c in mammalian subjects when other classical hemodynamic and metabolic measurements fail.

Various mechanisms have been proposed to explain how cytochrome c is released from the mitochondria. One mechanism involves opening of a high-conductance mega channel known as the mitochondrial permeability transition pore (MPTP). The pore forms by apposition of at least three major proteins including adenine nucleotide translocator, voltage dependent anion channel, and cyclophilin D enabling molecules up to 1.5 kDa to pass through the channel. MPTP opening in vitro causes mitochondrial swelling with disruption of the outer mitochondrial membrane and release of cytochrome c. Another mechanism involves selective permeabilization of the outer mitochondrial membrane without MPTP opening. Pro-apoptotic and anti-apoptotic proteins of the beta-cell leukaemia/lymphoma-2 (Bcl-2) family play a critical role given their capability for forming channel-like structures when pro-apoptotic members translocate to the outer mitochondrial membrane. Cytochrome c release is facilitated by concurrent peroxidation of cardiolipin by ROS. Peroxidation of cardiolipin decreases its binding affinity for cytochrome c creating a soluble pool that can be released to the cytosol.

The specific mechanism(s) responsible for cytochrome c release in our experimental model is not yet known. It is conceivable that a particular mechanism of cytochrome c release may relate to the type and severity of tissue injury. For instance, MPTP opening but not outer mitochondrial membrane permeabilization causes collapse of the mitochondrial voltage gradient leading to uncoupling of oxidative phosphorylation and cessation of ATP production.

The mechanism by which cytosolic cytochrome c reaches the extracellular space and appears in the bloodstream is not well understood in the art. Several studies have shown that cytochrome c release outside the cell occurs without concomitant release of larger molecules such as lactate dehydrogenase, which is considered a marker of cell necrosis with disruption of cell membrane. Regardless of mechanisms, the present invention demonstrates a progressive rise in plasma cytochrome c levels during the post-resuscitation period attaining levels that were inversely related to survival outcome. Increases in cytochrome c occurred after return of spontaneous circulation despite reversal of whole-body ischemia. This suggests that ischemia and reperfusion trigger processes leading to progressive rise in plasma cytochrome c after return of metabolically adequate blood flow. Organs rich in mitochondria with high metabolic rate and prone to ischemia and reperfusion injury are the heart and brain. However, without knowing the specific mechanisms of cytochrome c release especially after return of spontaneous circulation, other mitochondria-rich organs such as the liver, kidney, pancreas, and skeletal muscle cannot be excluded. The possibility that plasma cytochrome c was contributed by circulating blood cells was considered. Circulating red cells contain considerable amount of caspase-3; however, they lack nuclei, mitochondria, and the machinery for activation of apoptotic pathways. Neutrophils, on the other hand can undergo apoptosis. However, studies have shown that apoptosis in neutrophils is actually delayed under conditions such as acute respiratory distress syndrome, sepsis, and burn injury. According to the art there are no signs that apoptosis is activated in neutrophils at 4 hours post-reperfusion following tourniquet-induced ischemia in human skeletal muscle. Acute stress, in fact, seems to suppress the mitochondrial apoptotic pathway of neutrophils consequent to down-regulation of pro-apoptotic Bcl-2 proteins. Likewise, we found in the present studies that the percentage of neutrophils undergoing apoptosis is reduced after resuscitation from cardiac arrest and that increased levels of cytochrome c are not likely to induce apoptosis in leukocytes. Accordingly, the increased plasma levels of cytochrome c in the present studies most likely originated from organs that suffered ischemia and reperfusion injury during cardiac arrest and resuscitation.

### Example 7

### Experiments Assessing the Effects of EPO

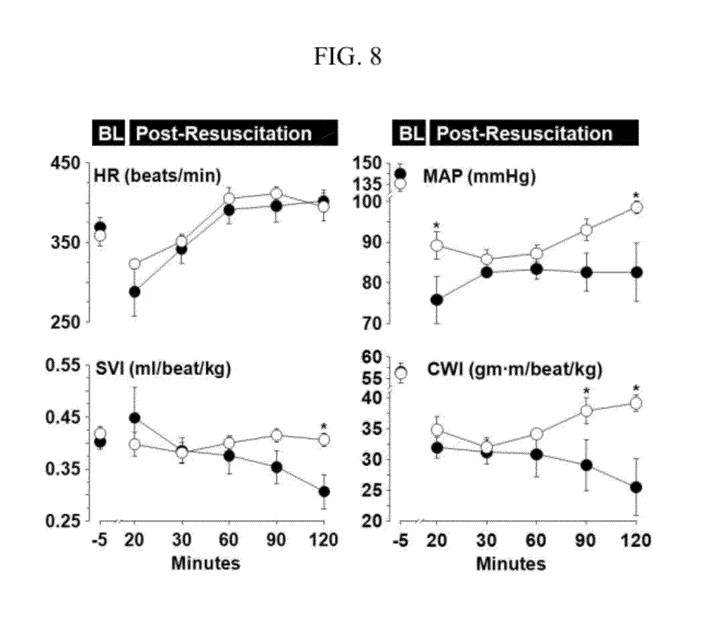

The effects of EPO given at the start of CPR on post-resuscitation myocardial function were further examined. Ventricular fibrillation was induced in 10 male adult Sprague-Dawley rats and left untreated for 8 minutes before attempting resuscitation by CPR and electrical shocks. Rats were randomized to receive EPO 5,000 U/kg (n=5) or saline vehicle (n=5) into the right atrium immediately before starting CPR with the investigators bind to the treatment assignment. All rats regained cardiac activity following CPR and were observed for 120 minutes post-resuscitation administering 0.9% NaCl intravenous (24 ml/kg/h) for preload support and dobutamine intravenous (15 μg/kg/min) for inotropic support. The results indicate that administration of EPO markedly improved post-resuscitation myocardial function evidenced by progressive increases in cardiac work index (CWI) as shown in FIG. 8. This effect was accompanied by increases in stroke volume index (SVI) and mean aortic pressure (MAP) with the maximal effect observed at 120 minutes post-resuscitation time at which the experiment was terminated and the hearts were removed storing the left ventricles at −80° C. Mitochondria were isolated from frozen tissue and cytochrome c oxidase (complex IV of the electron transport chain) activity was measured using a technique based on determining the oxidation rate of externally supplied reduced cytochrome c (absorbance at 550 nm). Cytochrome c oxidase activity was measured in unlysed and lysed mitochondria to determine outer mitochondrial membrane (OMM) integrity.

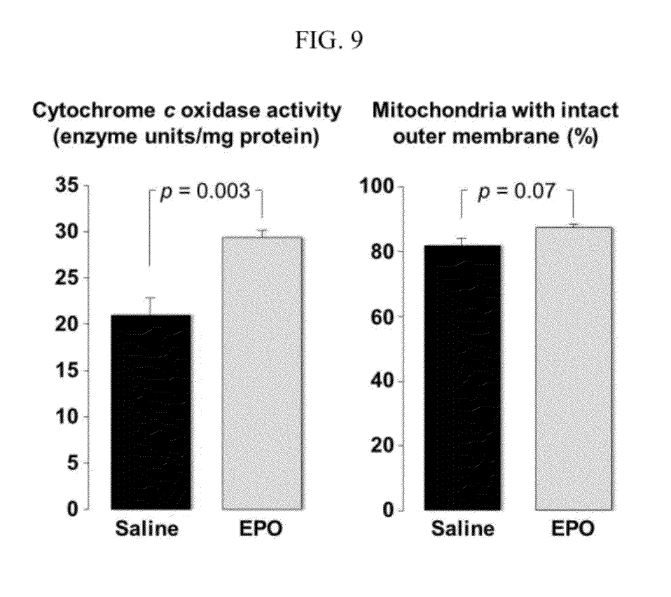

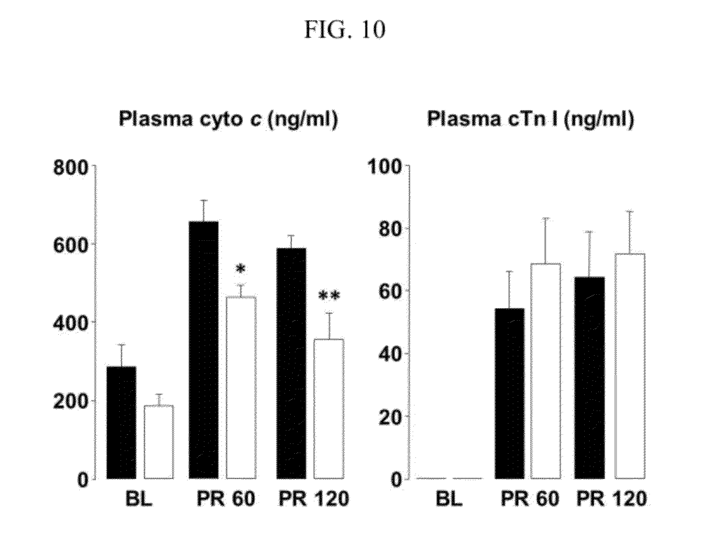

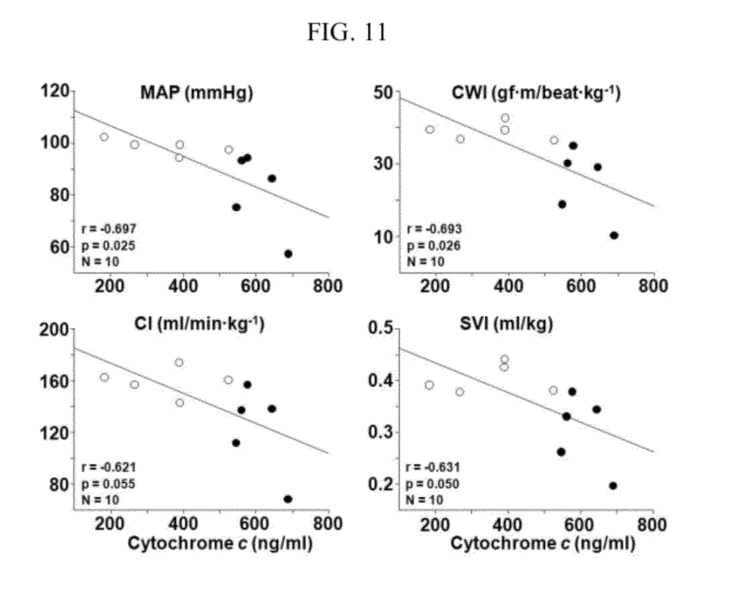

FIG. 9 shows that hearts treated with erythropoietin had a 40% higher cytochrome c oxidase activity than control hearts and higher percentage of mitochondria with intact outer mitochondrial membrane. These observations support the concept that erythropoietin protects mitochondrial function and anatomical integrity, leading to better bioenergetic function and better post-resuscitation myocardial function. This effect could be central to the protective mechanism of erythropoietin during resuscitation from cardiac arrest. The results further show that administration of erythropoietin while attenuating mitochondrial injury during reperfusion as evidenced by increased cytochrome c oxidase activity it also lowered the plasma levels of cytochrome c attaining statistically significant differences at 60 minutes and at 120 minutes post-resuscitation despite no significant differences in the cardiospecific troponin I (FIG. 10). Moreover, plasma cytochrome c was inversely correlated with cardiac work index (CWI, r=0.693; p=0.026) and mean aortic pressure (MAP, r=−0.697; p=0.025), and showed borderline statistical correlation with cardiac index (CI, r=−0.621; p=0.055) and stroke volume index (SVI, r=−0.631; p=0.050) at 120 minutes post-resuscitation (FIG. 11). Accordingly, the present data demonstrate that erythropoietin ameliorates post-resuscitation myocardial dysfunction in part by reducing mitochondrial injury and that this effect is reflected in blood by reductions in plasma cytochrome c. Such robust relationship is expected primarily because cytochrome c release to the bloodstream is not an epiphenomenon but rather a direct manifestation of a key pathogenic event during reperfusion leading to cell dysfunction and cell death. In other words, cytochrome c appears in blood only because it has been released from mitochondria, an event which in and of itself is detrimental to cell function and viability.

### Significance and Clinical Relevance

The present invention discloses that circulating levels of cytochrome c after resuscitation from cardiac arrest and other low flow states may serve as an in vivo marker of reperfusion injury and prognosticate survival and also assess the effects of interventions given to ameliorate mitochondrial injury during reperfusion. One aspect of the present invention is that measuring blood cytochrome c levels after resuscitation and stabilization from whole body ischemia may enable one to: 1) recognize and quantitate the severity of reperfusion injury, 2) prognosticate organ dysfunction and survival, 3) timely triage victims and institute specific medical treatments, and 4) assess the impact of novel therapies aimed at preventing reperfusion injury by targeting mitochondrial mechanisms of injury. Such applications are useful in both civilian and military emergency settings and therefore have broad clinical applications.

The novelty of the present invention links circulating levels of cytochrome c directly to mitochondrial injury. The relationship between circulating levels of cytochrome c and underlying organ injury and survival, is also related to previous studies in U.S. patent application Ser. Nos. 11/489,846 and 12/613,919, where mammalian subjects were administered effective amounts of EPO at the start of cardiac resuscitation. Those studies demonstrate that a single bolus dose of erythropoietin given at the start of CPR enhances the hemodynamic efficacy of chest compression by attenuating decreases in left ventricular myocardial distensibility; an effect that is associated with preservation of mitochondrial bioenergetic function. Clinical studies show that patients suffering out-of-hospital sudden cardiac arrest demonstrate that erythropoietin given at the start of CPR resulted in hemodynamically more effective CPR and higher resuscitation rates. In fact, the survival rates of the patients discharged from the hospital exceeded projections based solely on initial resuscitation rates suggesting that the beneficial effects of erythropoietin could extend beyond initial resuscitation and contribute to hemodynamic stability post-resuscitation.

Accordingly, the present invention supports the use of cytochrome c as a biomarker of reperfusion injury for use in examining the effects of treatments designed to attenuate mitochondrial injury expecting to observe a close relationship between effects on organ function, survival, and circulating levels of cytochrome c. The present invention uses EPO as an example of such mitochondrial targeted intervention. Other interventions likely to attenuate mitochondrial injury are also expected to result in functional benefits associated with attenuation in circulating levels of cytochrome c.

The present invention further discloses that the plasma levels of cytochrome c that correlate with left ventricular and hemodynamic function are below the typical level of detection of 1 to 2 μg/ml of currently available tests, such as those based on ELISA. The plasma levels of cytochrome c that correlated with left ventricular function in the present embodiments ranged from 0.2 to 0.8 μg/ml (FIG. 11). These levels can be detected by using HPLC and western blot technique as shown in FIGS. 1, 2 and 3. However, the levels that were associated poor survival exceeded 2 μg/ml (FIG. 7). Accordingly, optimal application of this invention enabling assessing effects of interventions on left ventricular and hemodynamic function as well as effects on survival requires a technique with broad dynamic range with a level of detection starting at least at 0.2 μg/ml and exceeding 8 μg/ml.

The present invention has clinical relevance for it is expected to provide the rationale and specification for developing rapid assays to measure cytochrome c in blood for assessing reperfusion injury and the effects of mitochondrial targeted interventions after resuscitation from cardiac arrest and other low flow states. Such assay could be run by emergency medicine providers enabling the use of cytochrome c levels to assess the probability of death triggering specific treatments and triage decisions, including priority access to a medical facility along with selection of victims with high cytochrome c levels dictates identification of unresolved precipitating events, completion of initial resuscitation efforts, and minimization of additional injury.

In addition, the capability for real-time recognition and severity assessment of mitochondrial-related reperfusion injury will enable clinical testing of novel treatments targeted at mechanisms of mitochondrial injury, such as the use of EPO.

Thus the present invention provides a method for determining the effectiveness of treatments for attenuating mitochondrial injury including the steps of: (1) administering to a mammalian subject having suffered whole body ischemia a treatment to attenuate mitochondrial injury; (2) measuring a plasma level of cytochrome c in the mammalian subject after resuscitation; and (3) comparing the plasma level of cytochrome c to a normal plasma level of cytochrome c of the mammalian subject; wherein a continuous increase in the plasma cytrochrome c level after resuscitation compared to the normal level is indicative of mitochondrial injury in a subject; and wherein a lesser increase in the plasma cytochrome c level after resuscitation when given an intervention aimed at ameliorating mitochondrial injury is indicative of efficacy in attenuating mitochondrial and reperfusion injury by such intervention.

While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

We claim:

1. A method for determining the effectiveness of treatments for attenuating mitochondrial injury, the method comprising:
administering to a mammalian subject having suffered whole body ischemia a treatment to attenuate mitochondrial injury;
measuring a plasma level of cytochrome c in the mammalian subject after resuscitation; and
comparing the plasma level of cytochrome c to a normal plasma level of cytochrome c of the mammalian subject; wherein a continuous increase in the plasma cytrochrome c level after resuscitation compared to the normal level is indicative of mitochondrial injury in a subject; and wherein a lesser increase in the plasma cytochrome c level after resuscitation when given an intervention aimed at ameliorating mitochondrial injury is indicative of efficacy in attenuating mitochondrial and reperfusion injury by such intervention.

2. The method of claim 1, wherein the treatment to attenuate mitochondrial injury is administering an effective amount of erythropoietin (EPO).

3. The method of claim 1, wherein the whole body ischemia is due to cardiac arrest or other hemodynamic crises.

4. The method of claim 2, wherein the hemodynamic crisis is hemorrhagic shock or hypovolemic shock.

5. The method of claim 1, wherein the mammalian subject is a human subject.
